(19) United States
(12) Patent Application Publication
(10) Pub. No.: US 2024/0293072 A1
(43) Pub. Date: Sep. 5, 2024

(54) MENTAL IMAGE-BASED NEUROFEEDBACK TO IMPROVE COGNITIVE FUNCTION

(71) Applicant: BROWN UNIVERSITY, Providence, RI (US)

(72) Inventor: Masahiro Machizawa, Providence, RI (US)

(21) Appl. No.: 18/578,379

(22) PCT Filed: Jul. 5, 2022
(86) PCT No.: PCT/US22/36103

§ 371 Date: Jan. 11, 2024

Related U.S. Application Data
(60) Provisional application No. 63/220,646, filed on Jul. 12, 2021.

(51) Int. Cl. A61B 5/375; A61B 5/378; A61B 5/384

(52) U.S. Cl. CPC A61B 5/375; A61B 5/378; A61B 5/384

(57) ABSTRACT

Neurofeedback training can include a subject visualizing a mental image, neural signals that quantify visual working memory related to the mental image can be recorded using scalp electrodes. The subject's performance with the visualization can be scored based on neural activity related to a cognitive function within the neural signals during an induction period. Feedback can be provided to the subject based on the scoring to inform the subject of a success rate related to the visual working memory. The goal of the neurofeedback training can be to improve the subject's cognitive function.

### CROSS-REFERENCE TO RELATED APPLICATIONS

This application claims the benefit of U.S. Provisional Patent Application No. 63/220,646, filed Jul. 12, 2021, entitled MENTAL IMAGE-BASED NEUROFEEDBACK TO IMPROVE COGNITIVE FUNCTION. The subject matter of this application is incorporated herein by reference in their entirety.

### GOVERNMENT SUPPORT

This invention was made with government support under grant numbers R01EY019466 and R01EY027841 awarded by the United States National Institutes of Health, grant number BSF 2016058 awarded by the United States-Israel Binational Science Foundation, and grant number JPMJCE1311 awarded by the Japanese Science and Technology Agency. The governments of the United States, Israel, and Japan have certain rights in the invention.

### TECHNICAL FIELD

The present disclosure relates generally to neurofeedback and, more specifically, to systems and methods that provide neurofeedback based on neural signals related to a mental image to improve at least one cognitive function.

### BACKGROUND

Neurofeedback can be used to modify brain function and structure through operant conditioning by measuring real-time brain activity and providing feedback to a user. Neurofeedback training has been employed in order to reduce symptoms of certain disorders (e.g., ADHD, depression, anxiety, traumatic brain injury, stroke, epilepsy, etc.) in cognitively disrupted patients and to enhance cognitive function (e.g., visual attention, working memory, concentration, short term memory, emotion, creativity, etc.) in cognitively healthy patients. Recently, neurofeedback training has gained prominence. Gameplay, specifically, has been advertised as an easy way to enhance cognitive performance. However, lengthy training with tasks using gameplay or other simple neurofeedback techniques has been shown not to robustly enhance cognitive functions that generalize to fundamental higher cognitive functions, and instead only improve the performance of the trained task.

### SUMMARY

As an alternative to behavioral training techniques, such as gameplay and other simple neurofeedback techniques, which do not improve cognitive functions that generalize to fundamental higher cognitive functions, the present disclosure provides systems and methods that employ a neurofeedback technique that does robustly improve cognitive functions that generalize to fundamental higher cognitive functions. The neurofeedback technique provided by the present invention is based on a user imagining (or mentalizing) a mental image, trying to improve a previous score based on the mental image, and providing a new score based on the mental image.

A system can be configured to engage a subject in a neurofeedback training trial. The system includes a memory storing instructions and a processor configured to access the memory and execute the instructions to: receive neural signals that quantify visual working memory related to a mental image from a plurality of scalp electrodes; score the subject's performance based on neural activity related to a cognitive function during an induction period within the neural signals; and provide feedback to the subject based on the scoring to inform the subject of a success rate related to the visual working memory. After the trial, the cognitive function of the subject improves.

A method for neurofeedback training can improve a cognitive function of a subject. The method includes engaging a subject in neurofeedback training over a time period. During each trial of the neurofeedback training: recording neural signals that quantify visual working memory related to a mental image using scalp electrodes; scoring the subject's performance based on neural activity related to a cognitive function during an induction period within the neural signals; and providing feedback to the subject based on the scoring to inform the subject of a success rate related to the visual working memory. The cognitive function of the subject improves after the time period.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is diagram of an example system that can provide neurofeedback based on neural signals related to a mental image to improve a cognitive function;

FIG. 2 is a diagram showing the different periods of neurofeedback training executed by the system of FIG. 1;

FIG. 3 is a process flow diagram of a method for executing a single trial of the neurofeedback training described herein;

FIG. 4 is a process flow diagram of a method for providing a score for the trial of FIG. 3;

FIG. 5 shows scalp topography of CDA channels and corresponding feedback scores for each group;

FIG. 6 is a schematic of the neurofeedback training trial;

FIG. 7 is a flow diagram of a real-time computations work-flow;

FIG. 8 shows scalp topography of averaged ERP over 400-1000 ms and 1000-5000 ms after the go-signal onset with gray circles indicating target channels of interest;

FIG. 9 shows the grand-averaged hemispheric differential ERP waveforms (left hemisphere-right hemisphere electrodes) of each group over 400-1000 ms and 1000-5000 ms;

FIG. 10 shows the grand averaged hemispheric differential amplitude for the CDA channels over training sessions (days 1-5);

FIG. 11 shows attentional efficiency scores and visual working memory scores for pre- and post-training sessions; and

FIG. 12 shows working memory increase was associated with the degree of left-lateralized CDA and behavioral change was not correlated with the degree of right-lateralized CDA.

### DETAILED DESCRIPTION

### I. Definitions

Unless otherwise defined, all technical terms used herein have the same meaning as commonly understood by one of ordinary skill in the art to which the present disclosure pertains.

As used herein, the singular forms “a,” “an” and “the” can also include the plural forms, unless the context clearly indicates otherwise.

As used herein, the terms “comprises” and/or “comprising,” can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

As used herein, the terms “first,” “second,” etc. should not limit the elements being described by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

As used herein, the term “neurofeedback” can refer to a form of biofeedback in which a user reacts to feedback (e.g., sensory feedback), often in real-time to a brain activity.

As used herein, the term “neurofeedback training” can refer to the use of neurofeedback to modify a brain function or structure through operant conditioning. The operant conditioning can be based on feedback (e.g., sensory feedback) that facilitates self-regulation. For example, neurofeedback training has been employed in order to reduce symptoms of certain disorders (e.g., ADHD, depression, anxiety, traumatic brain injury, stroke, epilepsy, etc.) and to enhance cognitive function.

As used herein, the term “cognitive function” is a broad term that can refer to mental processes involved in the acquisition of knowledge, manipulation of information, and reasoning. Cognitive functions can include, for example, visual attention, working memory (also referred to as visual working memory), concentration, short term memory, emotion, creativity, etc.

As used herein, the term “mental image”, “mental picture”, “mental imagery”, or the like, can refer to a sensory experience that can resemble an experience of perceiving some object, event, and/or scene, but occurs when the relevant object, event, or scene is not actually present to the senses. In other words, a mental image can be “imagined”, “created”, “mentalized”, or the like with or without the exertion of effort.

As used herein, the terms “user”, “participant”, “subject”, or the like can refer to any human being that has engaged or will engage in neurofeedback training.

As used herein, the term “controller” can refer to anyone performing neurofeedback training and may include one or more computing devices and/or a human executing the neurofeedback training.

As used herein, the term “real-time” can refer to a time in which feedback becomes available after an event. In some instances, feedback can be real-time if it becomes available within 10 seconds after a certain event. In other instances, feedback can be real-time if it becomes available within 5 seconds after a certain event. In still other instances, feedback can be real-time if it occurs within 1 second after a certain event. In still other instances, feedback can be real-time if it occurs within 50 milliseconds after a certain event.

### II. Overview

Traditionally, gameplay and other simple neurofeedback techniques have been touted as treatments that can enhance cognitive function. However, gameplay and other simple neurofeedback techniques have been shown not to robustly enhance cognitive function or generalize to higher cognitive function. As an alternative to gameplay and other simple neurofeedback techniques, the present disclosure provides systems and methods that employ a neurofeedback technique based on a user imagining a mental image to improve cognitive function. The neurofeedback technique described herein takes advantage of humanity's ability to learn and accumulate statistical experience and thereby improves one or more cognitive function. Specifically, the neurofeedback technique includes one or more trials, in which neural signals are recorded that quantify visual working memory related to a mental image using scalp electrodes (placed on a user doing the neurofeedback training), the user's performance is scored based on neural activity related to a cognitive function during an induction period within the neural signals, and feedback is provided to the user based on the scoring to inform the subject of a success rate related to the visual working memory. The user aims to improve the score across different trials. The cognitive function is improved after the one or more trials.

### III. Systems

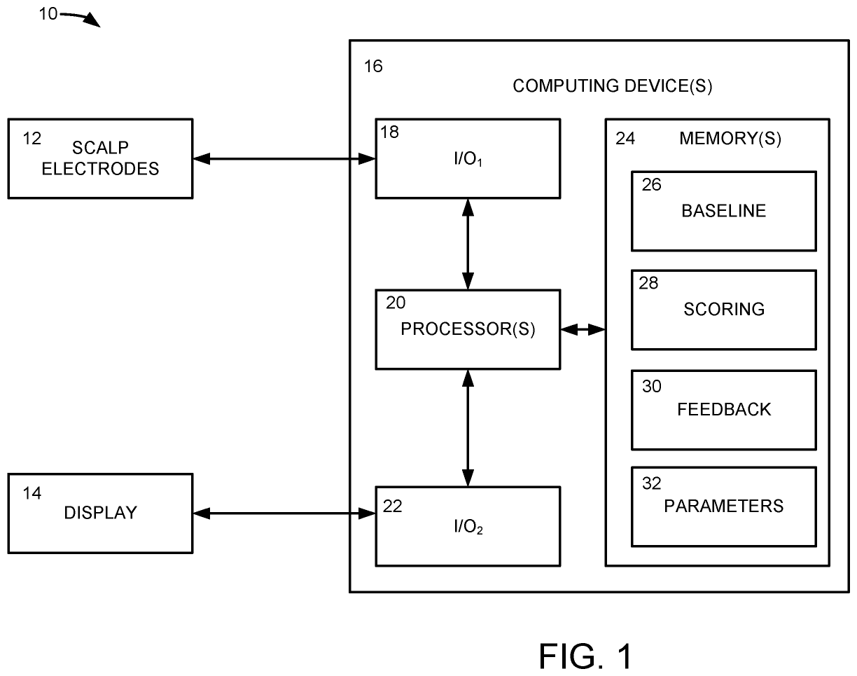

An aspect of the present disclosure can include a system 10 (FIG. 1) that can provide neurofeedback based on neural signals related to a user forming a mental image. The mental image can be created/formed by the user without being prompted about what to imagine. For example, the mental image can be based on a memory specific to the user. The mental image can be, but does not have to be, different for every different user and/or for every neurofeedback training trial. For example, one user may form a mental image of an auditory sound (e.g., hearing their mother's voice), while another may form a visual mental image of a sunny day at a beach. In other words, the specific mental image does not matter, but the fact that the user is creating a mental image is the subject of the neurofeedback. The neurofeedback provided by the system of FIG. 1 has been shown to enhance one or more cognitive functions and is superior to that provided by gameplay and other simple neurofeedback techniques, which have been shown not to robustly enhance cognitive functions.

A user can engage in neurofeedback training, which can include one or more trials, using the system of FIG. 1. It will be understood that while the system 10 is described for the execution of a single neurofeedback training trial, system 10 can be used to conduct one or more neurofeedback training trials (e.g., as described in the Experimental section below).

The system 10 can include scalp electrodes 12, a display 14, and one or more computing devices 16. The scalp electrodes 12 can be connected to the one or more computing devices 16 through an input/output port (I/O₁) 18, while the display 14 can be connected to the one or more computing devices 16 through another input/output port (I/O₂) 22. Each of the input/output ports 18, 22 can include circuitry configured to facilitate data communication/transfer between the one or more computing devices 16 and a device (e.g., scalp electrodes 12 or display 14) hooked to the respective input/output port (this circuitry may be the same or different in the different ports). Moreover, each of the respective input/output port may be configured for wired and/or wireless communication. Additionally, it will be understood that the one or more computing devices 16 may have more than two ports.

The scalp electrodes 12 can be configured to be placed and positioned at predetermined locations on the subject's scalp to record neural signals. The scalp electrodes 12 can be active electrodes and/or passive electrodes. As one example, the scalp electrodes 12 can be EEG electrodes (e.g., traditional wet Ag/AgCl electrodes, active dry single gold pin-based electrodes, hybrid dry multiple spikes-based electrodes, passive dry solid-gel based electrodes, etc.). A comprehensive standard EEG analyses use as many as 64 electrodes. The system 10 may also use as many as 64 scalp electrodes 12, but may instead utilize only a portion of the EEG electrodes traditionally used (e.g., utilizing only posterior-parietal EEG electrodes and/or occipital EEG electrodes). Each of the scalp electrodes 12 can be associated with a channel (e.g., the scalp electrodes can include electrodes positioned posteriorly and parietally that are associated with one or more posterior parietal channel and/or electrodes positioned near the occipital lobe that are associated with one or more occipital channels). As an example, in some instances, the neural signals can be measured from at least one of the one or more posterior parietal channels and/or at least one of the one or more occipital channels. The scalp electrodes 12 can transmit the neural signals to the one or more computing devices 16 through the one or more channels connected to the input/output port (I/O₁) 18. It will be understood that the computing device 16 and/or one or more additional components associated with the scalp electrodes 12 may perform pre-processing tasks on the neural signals recorded by the scalp electrodes 12.

The display 14 can display visual signals, auditory signals, tactile signals, or the like to the user during neurofeedback training. The display 14 can include a monitor (e.g., capable of providing a visual stimulus), a speaker (e.g., capable of providing an audio stimulus), and/or a different peripheral device (e.g., capable of providing a tactile stimulus). For example, the display 14 can display an indication for a user to start and/or stop a neurofeedback training trial or an indication of feedback, such as a score, related to the neurofeedback training trial. The display 14 can send/receive data to/from the one or more computing devices 16 through input/output port (I/O₂) 22.

The one or more computing devices 16 can include the input/output ports 18, 22 (and any additional ports) that are connected for data communication with one or more processors 20. Additionally, the one or more processors 20 can also be connected for data communication with one or more non-transitory memory devices. As an example, the one or more processors 20 and the one or more non-transitory memory devices 24 The one or more computing devices 16 may also have additional components that are not illustrated that are also connected for data communication with one or more processors 20.

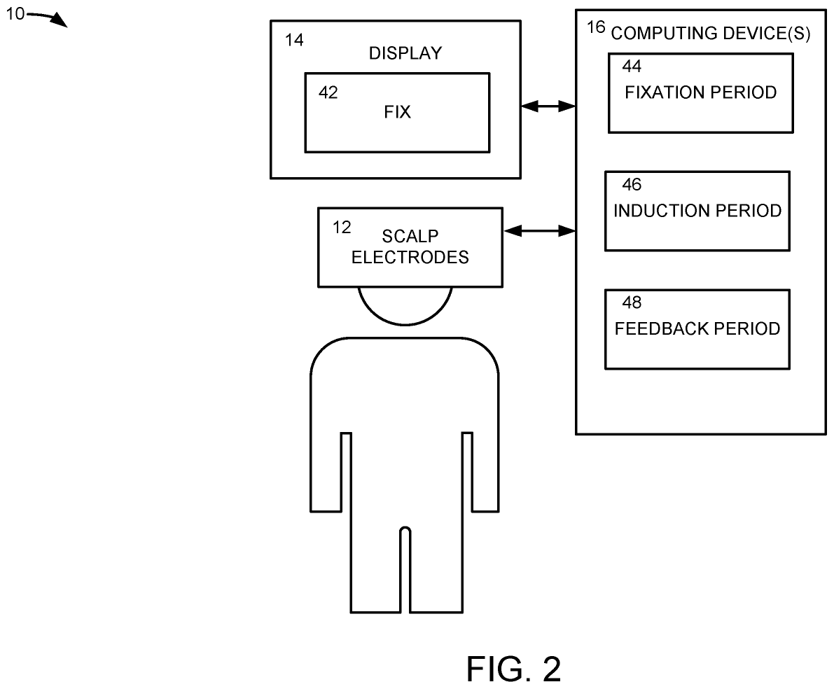

The one or more computing devices 16 can be programmed for one or more processors 20 to execute a plurality of tasks (shown, for example, in FIG. 1 and FIG. 2) based on instructions stored in one or more non-transitory memory devices 24. The one or more computing devices 16 can be implemented as a desktop computer, a portable computer (e.g., a laptop, a smart phone, a tablet, etc.), or the like. The one or more computing devices 16 can act as a controller (either with human intervention or autonomously) to perform the neurofeedback training. Additionally, it should be noted that the one or more computing devices 16 are not limited to only the components previously described, the one or more computing devices may have additional components that are not illustrated.

The one or more non-transitory memory devices 24 can be configured to store machine readable instructions and/or data. The one or more non-transitory memory devices 24 can be implemented, for example, as volatile memory (e.g., RAM), nonvolatile memory (e.g., a hard disk, flash memory, a solid state drive or the like) or combination of both. The one or more processors 20 (e.g., one or more processor core) can be configured in the system for accessing the one or more non-transitory memory devices 24 and executing the machine-readable instructions.

By way of example, the one or more non-transitory memory devices 24 can store a variety of machine-readable instructions and data, including an operating system, one or more application programs, other program modules, and program data. The operating system can be any suitable operating system or combinations of operating systems, which can depend on manufacturer and system to system. In some examples, the application programs and program modules can implement part, or all, of the neurofeedback training. For example, the program modules can include a baseline module 26, a scoring module 28, and a feedback module 30 (the system 10 can have more program modules than those illustrated). Additionally, the non-transitory memory 24 can store parameters 32 (e.g., data, settings, or the like regarding the user and/or the neurofeedback training trial) or other information related to the user and/or the neurofeedback training trial. As an example, the parameters 32 can include adjustment parameters for the user's score.

As shown in FIG. 2, the neurofeedback training trial using the system 10 can include a fixation period 44, an induction period 46, and a feedback period 48. The baseline module 26 can be executed during the fixation period 44, the scoring module 28 can be executed during the induction period 46, and the feedback module 30 can be executed during the feedback period 48. The program modules may also be executed, partially or fully during different periods than those just described.

The fixation period can be when the user fixes on a fix 42 point on the display 14. The fix 42 point may be a visual image, like a shape on a monitor (display 14), for example. However, the fix point 42 need not be a visual image. The fixation period can be, for example, 20 seconds or less, 10 seconds or less, or 5 seconds or less. A baseline score can be determined by the baseline module 26 during the fixation period 44 (e.g., based on data recorded by the scalp electrodes 12 during the fixation period and/or based on saved data; the data may be pre-processed). Alternatively, or additionally, the baseline module 26 can determine the baseline score adaptively during at least a portion of the induction period 46. The baseline module 26 can determine the baseline score based on the user's neural activity when the user is not actively creating the mental image. In some instances, the baseline score can be set (e.g., by the scoring module 28) to a zero value and the score can be adjusted based on the zero value. It should be noted that the fixation period 44 and the induction period 46 need not be distinct periods occurring sequentially; instead, the fixation period 44 may occur during the induction period 46.

The induction period 46 can occur after the fixation period 44 (e.g., 5 seconds or less), but the fixation period 44 can, alternatively, be within the induction period 46. During the induction period 46, the user can create (or mentalize) a mental image and the scalp electrodes 12 can record the neural signals of the user as the user is creating/metalizing the mental image. The mental image can be a mental sensory experience that may resemble an experience of the user, such as perceiving some object, event, and/or scene. The mentally perceived object, event, and/or scene is not actually present to the senses during the neurofeedback training trial. It should be noted that an individual user's mental image need not be the same as another user's mental image. It should also be noted that when one user completes more than one neurofeedback training trial the user's mental image can be, but does not have to be, different during one or more of the trials. Additionally, in most instances, the system 10 provides no prompt to the user regarding the content of the mental image (or even whether the mental image should be auditory, visual, tactile, or the like). However, in some instances, the system 10 can indicate on the display that the user should start/stop creating the mental image. Additionally, during the induction phase 47, a target feature can be computed; the target feature can be within the neural signals from the scalp electrodes 12 and can be used to determine/adjust the score.

During the induction period 46 the user's performance at constructing the mental image can be scored (e.g., by scoring module 28) As an example, the score can be determined relative to the baseline based on changes in an aspect of the neural activity recorded by the scalp electrodes 12 (e.g., one or more slow cortical potentials). Slow cortical potentials are generally event-related shifts in potential related to cortical electrical activity that can be seen in a neural signal lasting from several hundred milliseconds to several seconds, like contralateral delay activity (CDA). However, slow cortical potentials other than CDAs may be used based on the cognitive function(s) targeted for improvement. For example, one or more slow cortical potentials in the neural activity can be reflective of bilateral or unilateral hemispheric activity, or bilateral differences between the hemispheres. The scoring includes comparing the neural activity related to the cognitive function during the induction period to a baseline neural activity. Please note that the baseline may be based on the same one or more slow cortical potentials as the score.

After the score is calculated (e.g., by scoring module 28), feedback is displayed via display 14 (e.g., in a visual manner, an auditory manor, and/or a tactile matter) during the feedback period 48 (e.g., by feedback module 30). The feedback can be illustrative of the success related to the specific cognitive function. The feedback can be a representation of the score (e.g., a good score can have a positive graphical indication and/or a positive auditory indication and bad score can have a negative graphical indication and/or a negative auditory indication). For example, the score can be shown on the display 14 (and may include an indicator of whether the score is good or bad). As an example, the score can be displayed less than 5 seconds after neural signals are recorded. As another example, the score can be updated at an interval (e.g., a predefined interval) during the trial to encourage the user to create a better mental image (or improve the mental image). The score may be adjusted during the trial to inform the user of progress. When the neurofeedback training includes more than one trial, the feedback period 48 can include the feedback module 30 instructing and/or encouraging the subject to improve the score in the future. The goal of the neurofeedback training is to improve a cognitive function of the user (the cognitive function may improve regardless of the user's success during the particular trial).

### IV. Methods

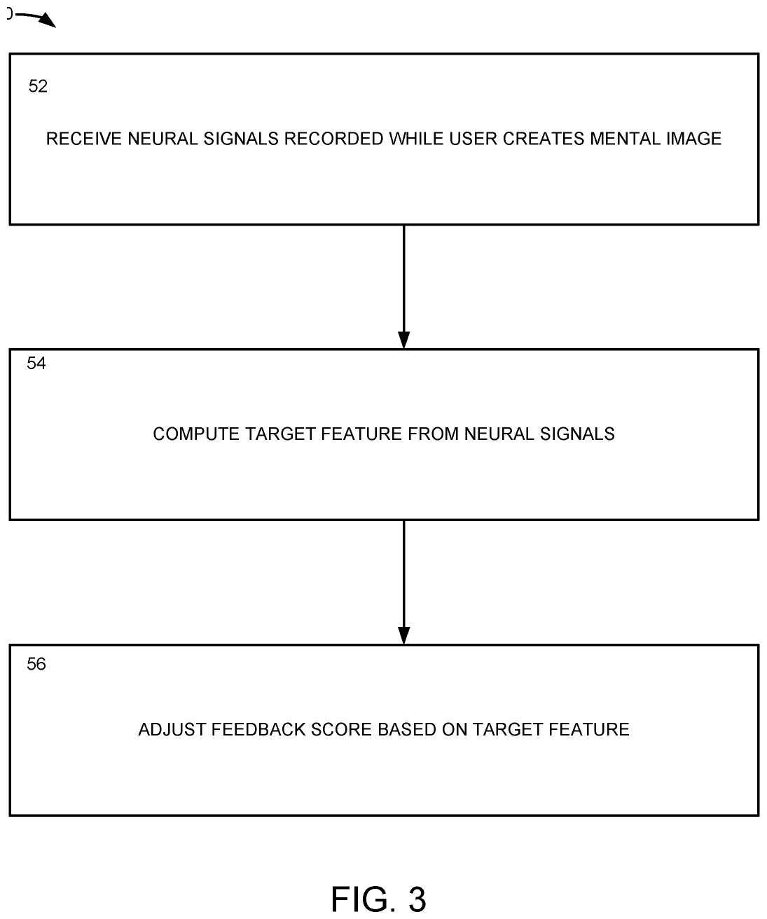

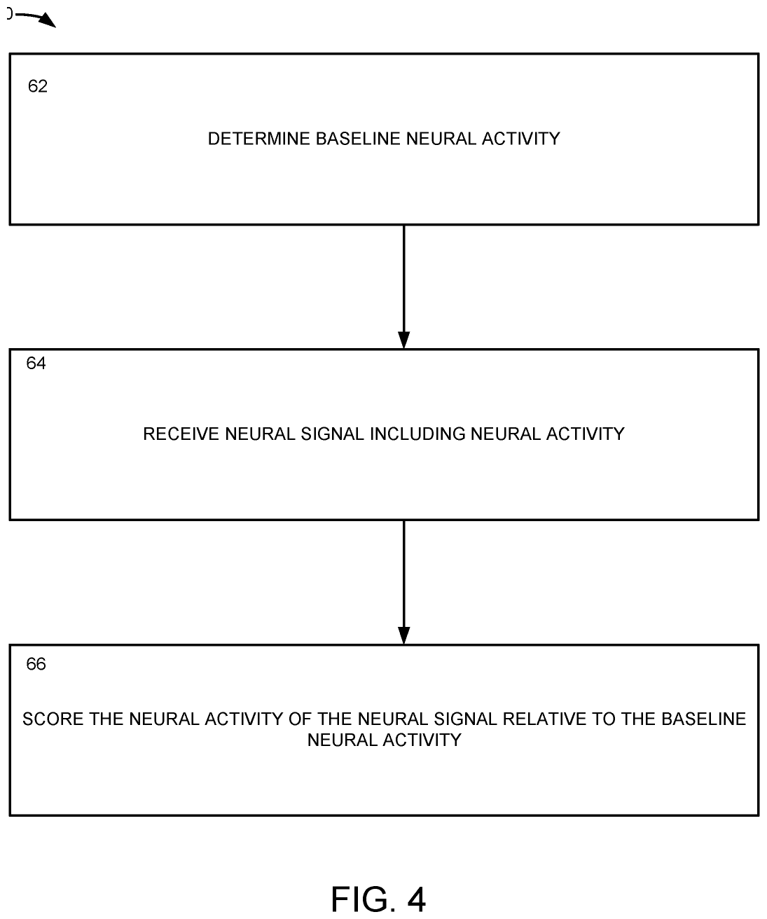

Another aspect of the present disclosure can include methods 50 and 60 (FIGS. 3 and 4) for providing neurofeedback training based on neural signals related to a mental image. The methods 50 and 60 can be executed using the system 10 shown in FIGS. 1 and 2. Specifically, one or more steps of methods 50 and 60 can be stored in the memory 24 and executed by the processor 20.

For purposes of simplicity, the methods 50 and 60 are shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the methods 50 and 60, nor are methods 50 and 60 limited to the illustrated aspects.

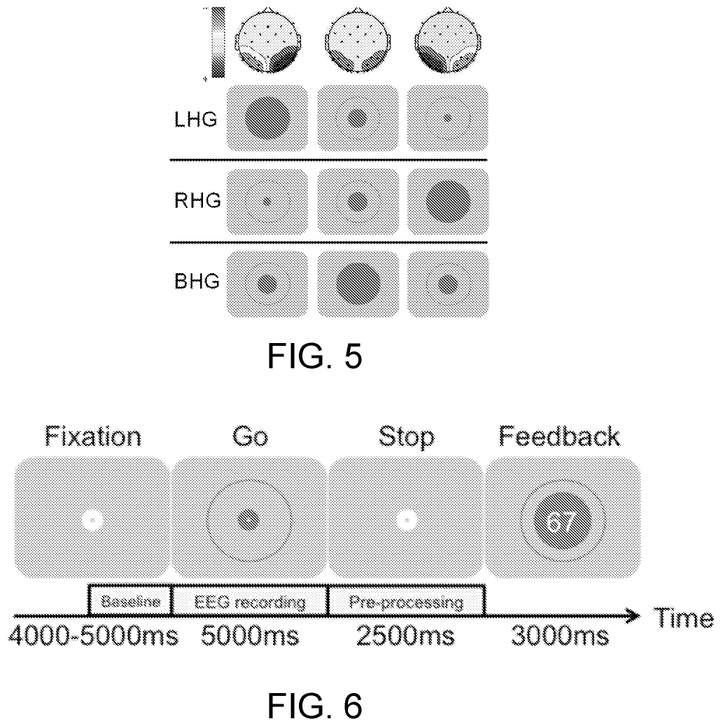

Referring now to FIG. 5 illustrated is a method 50 for executing a single trial of the neurofeedback training described herein. This method 50 can be executed multiple times during a time period until the neurofeedback training is complete. The time period can be on the order of minutes, hours, days, weeks, months, or years. The goal of method 50 is to improve a certain cognitive function. Cognitive functions can include, for example, visual attention, working memory (also referred to as visual working memory), concentration, short term memory, emotion, creativity, etc.

At 52, neural signals that are recorded (e.g., by scalp electrodes 12) while a user creates a mental image can be received (e.g., by computing device 16). The neural signals can quantify visual working memory, and/or another cognitive function, related to a mental image. The scalp electrodes can be positioned on a user's scalp and may include active electrodes or passive electrodes. The scalp electrodes may be linked to one or more posterior parietal channels and/or one or more occipital channels (e.g., EEG channels).

At 54, a target feature (e.g., of the neural signals) can be computed from the neural signals. For example, the target feature can be related to the target cognitive function that is trying to be improved by the neurofeedback training. At 56, a feedback score can be adjusted based on the target feature (e.g., by the feedback module 30 of the computing device 16)

The user's performance can be scored (e.g., by scoring module 28 on computing device 16 with the adjusted feedback score) based on neural activity within the neural signals. The scoring can occur during an induction period and may be based on a baseline score (determined during a fixation period or at a time during the induction period when the user is not creating, or trying to create, the mental image). The induction period may follow the fixation period, but the fixation period also may be within the induction period. During the induction period, the user can create (or mentalize) a mental image and neural signals can be recorded from the scalp electrodes. The mental image can be a sensory experience that can resemble an experience of the user perceiving some object, event, and/or scene, but occurs when the relevant object, event, or scene is not actually present to the senses. The mental image can be created/formed by the user without being prompted about what to imagine. For example, the mental image can be based on a memory specific to the user. The mental image can be, but does not have to be, different for every different user and/or for every neurofeedback training trial. For example, one user may form a mental image of an auditory sound (e.g., hearing their mother's voice), while another may form a visual mental image of a sunny day at a beach. In other words, the specific mental image does not matter, but the fact that the user is creating a mental image is the subject of the neurofeedback. It should also be noted than an individual user's mental image may change between multiple trials of neurofeedback training. No prompt is provided to the user regarding the content of the mental image, but a prompt may be provided to instruct the user to start/stop creating the mental image.

The feedback can be provided to the user (e.g., via display 14) to inform the user of their success rate related to the mental image. The feedback can be illustrative of the user's success at creating a mental image and can be related to improvement of a specific cognitive function. For example, the score can be presented by the display 14 (and may include an indicator of whether the score is good or bad). The score can be displayed in a visual fashion, an auditory fashion, a tactile fashion, or the like. As an example, the score can be displayed after neural signals are recorded. As another example, the score can be updated at an interval (e.g., a predefined interval) during the trial to encourage the user to improve their mental imagining in that trial. After each trial of the neurofeedback training, the subject can be instructed to improve the score in the future. The goal of the neurofeedback training is to improve a cognitive function of the user (which may occur regardless of the success of a single trial).

Referring now to FIG. 6, illustrated is an example method 60 for providing a score for a single trial of neurofeedback training, such as the trial illustrated in FIG. 3. It should be understood that different/additional steps may be necessary to compute the score. At 62, baseline neural activity can be determined (e.g., by the baseline module 26 of computing device 16). The baseline can be determined during a fixation period. The fixation period can be when the user fixes on a point (or other type of visual image, but may not be a visual image) on the display (e.g., fix point 42 on display 14). The fixation period can be, for example, 20 seconds or less, 10 seconds or less, or 5 seconds or less. A baseline score can be determined during the fixation period or adaptively during at least a portion of the induction period. The baseline score can be based on the user's neural activity when the user is not actively creating the mental image. In some instances, the baseline score can be set to a zero value and the score can be adjusted based on the zero value. It should be noted that the fixation period and the induction period nay be distinct periods occurring sequentially, but need not be distinct periods occurring sequentially; instead, the fixation period may occur during the induction period.

At 64, a neural signal indicating neural activity can be received (e.g., by I/O₁ 18 of computing device 16). During the induction period, the user can create (or mentalize) a mental image and the scalp electrodes (e.g., scalp electrodes 12) can record the neural signals of the user as the user is creating/metalizing the mental image. The neural signal can be pre-processed before further action occurs. The mental image can be a mental sensory experience that may resemble an experience of the user, such as perceiving some object, event, and/or scene. The mentally perceived object, event, and/or scene is not actually present to the senses during the neurofeedback training trial. It should be noted that an individual user's mental image need not be the same as another user's mental image. It should also be noted that when one user completes more than one neurofeedback training trial the user's mental image can be, but does not have to be, different during one or more of the trials.

At 66, the neural activity of the neural signals can be scored (by scoring module 28 of computing device 16) relative to the baseline neural activity. The scoring can occur, for example, during the induction period, and can include comparing the neural activity related to the cognitive function during the induction period to a baseline neural activity. As an example, the score can be determined relative to the baseline based on changes in an aspect of the neural activity (e.g., one or more slow cortical potentials). Slow cortical potentials are generally event-related shifts in potential related to cortical electrical activity that can be seen in a neural signal lasting from several hundred milliseconds to several seconds, like contralateral delay activity (CDA) (however, other slow cortical potentials may be used based on the cognitive function targeted to improve). For example, one or more slow cortical potentials in the neural activity can be reflective of bilateral or unilateral hemispheric activity, or bilateral differences between the hemispheres.

### IV. Experimental

The following experiment demonstrates electroencephalogram (EEG)-based visual imagery neurofeedback training that can be used by the systems and methods described herein. The neurofeedback training can enhance one or more core cognitive functions, including higher cognitive functions thought to be common across various cognitive abilities. One such core cognitive function is working memory capacity (specific to attention and memory function) and a neural signature reflective of working memory capacity, contralateral delay activity (CDA), a slow event related potential (ERP) component that is a well-established ERP marker and is found within data of an EEG reading, but has not yet been used in real time neurofeedback training. Additionally, the neurofeedback training can change cortical anatomy connected to the enhanced cognitive function.

### Procedures:

Study participants (n=14), also referred to as users, were assigned to one of two groups randomly. One group (n=7) was trained on the left-hemisphere dominant CDA (‘LHG’), and the other group (n=7) was trained on the right-hemisphere dominant CDA (‘RHG’) (FIG. 5). Please note that, as shown in FIG. 5, bilateral dominant CDA (‘BHG’) was not explored but showing for a sample use-case purpose.

Each group participated in 100 trials of neurofeedback training (5 days, 20 trials per day). The following example takes advantage of the human ability to learn and accumulate statistical experiences about neural activity over the trials and days of the neurofeedback training. Without the neurofeedback training it is typically difficult to be aware of neural activity. Each day, each participants' task was to achieve a high score in as many trials as possible. The learning (e.g., the understanding of the state of brain activities) was on a trial-and-error basis. At the end of day, participants were reminded which trials had been successful (success was measured as a feedback score above 50, corresponding to more than 1 standard deviation relative to a baseline period for the particular participant). Each participant was asked what imagery they mentalized for that trial. Participants' task was to find their own strategy to achieve high scores (scores ranged from a low of 0 to a high of 100) in as many trials as possible, especially at the final training session.

As shown in FIG. 6, each trial was split into a fixation stage (lasting 4000-5000 ms, in order to determine a baseline), a go stage (lasting 5000 ms, when EEG was recorded), a stop stage (lasting 2500 ms, when pre-processing occurred), and a feedback stage (lasting 3000 ms, when a score was provided for the trial). During the fixation stage, participants were asked to fixate their eyes on a fixation point at the center of a circle. During the go stage, the participants were asked to close their eyes and form mental imagery (or “mentalize”) of anything they wanted until the stop stage where participants were free to open their eyes and stop mentalizing. In order to minimize any bias related to the mental imagery, the participants were told only that the target neural activity was believed to be associated with visual attention. During the go stage, high-quality EEG data was collected from 32 channel active electrodes (Brain Products, actiCAP, BrainAmp Standard Amplifier) covering the whole scalp of each participant and the CDA component of the EEG data was computed in real-time while participants concentrated on building mental imagery. The CDA component was then quantified and the score (from 0 to 100) was visualized on the screen in the feedback stage.

### EEG Procedures:

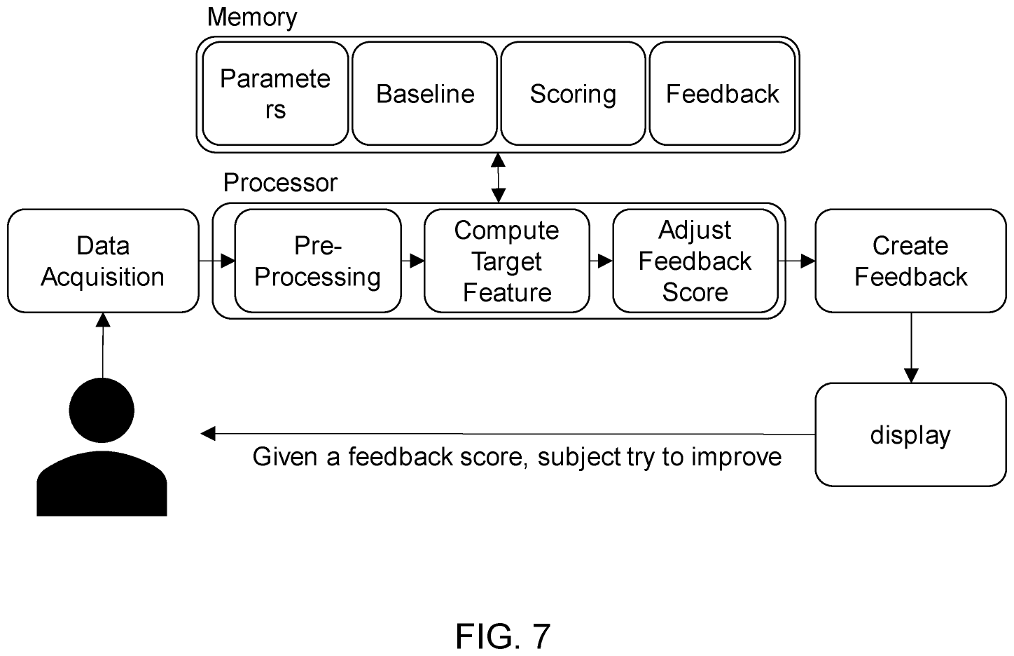

FIG. 7 shows an analytical workflow followed by this study. EEG signals were continuously monitored using BrainAmp Standard amplifier with 32 channel actiCAP electrodes from Brain Products GMBH and extracted into a PC in real time (at 500 Hz) throughout each trial using the Remote Data Access (RDA) TCP/IP protocol by Brain Products GMBH. For each trial, single-trial ERPs (event-related potentials) relative to the onset of the “Go” array were calculated from the posterior parietal and lateral occipital channels (namely, P5/6, P7/8, PO3/4, PO7/8, and O1/2). At the signal-offset, the raw EEG data was low-pass filtered at 30 Hz using an inverse Fast Fourier transform filter (without a high-pass filter) and then re-referenced to the mastoids and normalized to the baseline-period (the baseline-period being 5000 ms prior to the onset of the go-signal). Any channels with excessive amplitude changes (>100 μV/sec) within a trial were excluded. Then an averaged ERP was obtained for each hemisphere (one average ERP for left hemispheric channels and another average ERP for the right hemisphere). The absolute amplitudes of the resulting average ERPs were converted into Z-scores by normalizing the absolute amplitudes with the average of all corresponding channels' activities during the baseline period (baseline period=−500 to 0 ms relative to the go signal onset). For each hemisphere, the Z-converted amplitudes from the Go stage were averaged throughout the electrode array (Go stage=0-5000 ms after cue-onset), then the averaged Z-converted amplitudes were averaged for all corresponding channels for each hemisphere. The Z-score difference between the two hemispheres was calculated and the Z-score was converted into a percentile using a normal cumulative distribution function. When the Z-score for a target hemisphere was larger (more negative) than the other hemisphere, a larger circle was presented to the participant. When the score for the target hemisphere was smaller (more positive) than the other hemisphere, a smaller circle was presented to the participant. The diameters of the circle ranged from 1.6° to 20° visual angles. The size of the circle and the visualization of the score were modulated such that it would make it obvious for participants to know when they had a higher score, so as to encourage trials that produced larger CDAs and to discourage trials where bilaterally equal CDA amplitudes or smaller were found.

### Results

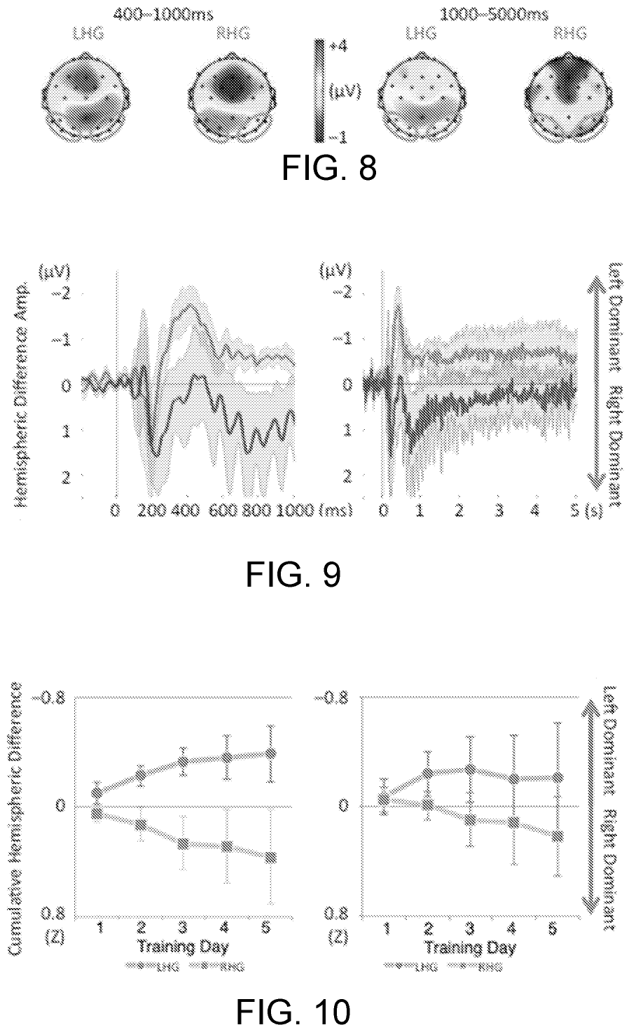

FIG. 8 shows EEG responses during the neurofeedback trials. Grand-averaged differential ERPs (FIG. 9) suggest each group successfully produced their CDAs in a congruent manner as was intended. Left Hemisphere group (‘LHG’) successfully produced CDAs toward the left-oriented direction after at around 3-400 msec. The induced CDAs improved over training sessions (FIG. 10). These results indicate the CDA neurofeedback was successful on an overall group-level.

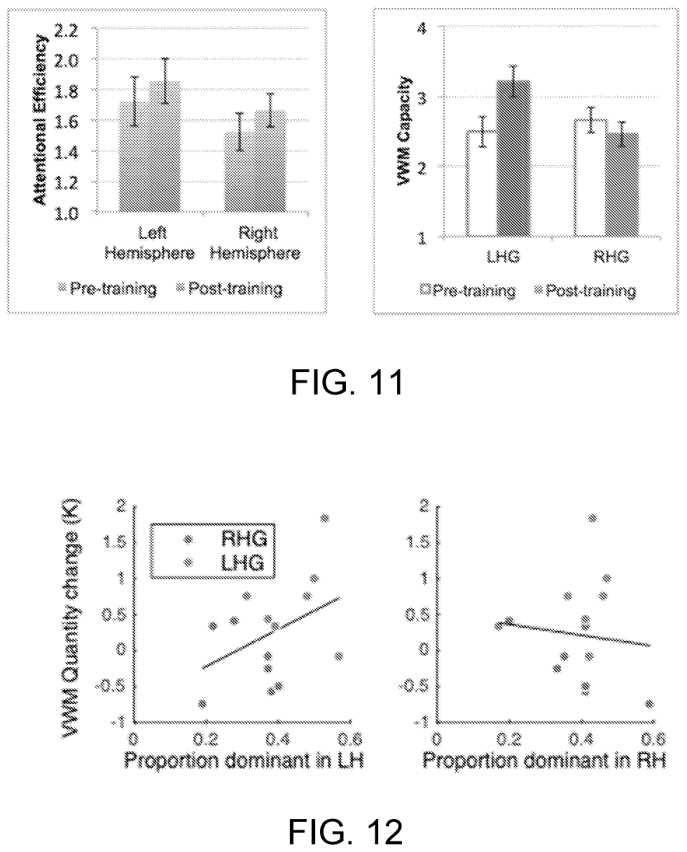

The individual user's attentional and working memory performance was assessed before and after the CDA neurofeedback training. Following the intensive training over five days, the participants significantly improved their ability to concentrate (attentional efficiency) regardless of the assigned group (FIG. 11, left), and most importantly, the method robustly improved working memory capacity with a high effect-size (Cohen's d, 0.94; FIG. 11, right) that has not been possible previously. Additionally, in FIG. 12, the working memory increase was associated with the degree of left-lateralized CDA (left), and the behavioral change also was not correlated with the degree of right-lateralized CDA (right).

From the above description, those skilled in the art will perceive improvements, changes and modifications. Such improvements, changes and modifications are within the skill of one in the art and are intended to be covered by the appended claims.

The following is claimed:

1. A method comprising:
engaging a subject in neurofeedback training over a time period;
during each trial of the neurofeedback training:
recording neural signals that quantify visual working memory related to a mental image using scalp electrodes;
scoring the subject's performance based on neural activity related to a cognitive function during an induction period within the neural signals; and
providing feedback to the subject based on the scoring to inform the subject of a success rate related to the visual working memory;
wherein the cognitive function of the subject improves after the time period.

2. The method of claim 1, further comprising during each trial of the neurofeedback training, positioning the scalp electrodes on the subject's scalp.

3. The method of claim 2, wherein the scalp electrodes comprise active electrodes and/or passive electrodes.

4. The method of claim 2, further comprising establishing channels for the scalp electrodes, wherein the channels comprise one or more posterior parietal channels and/or one or more occipital channels.

5. The method of claim 4, wherein the neural signals are measured from at least one of the one or more posterior parietal channels and/or at least one of the one or more occipital channels.

6. The method of claim 1, wherein the scoring comprises comparing the neural activity related to the cognitive function during the induction period to a baseline neural activity.

7. The method of claim 6, wherein the baseline neural activity is determined during a fixation period before the induction period or determined during the induction period in an adaptive manner.

8. The method of claim 7, wherein the fixation period is 5 seconds or less before the induction period, and
wherein the induction period is 5 seconds or less.

9. The method of claim 6, wherein the baseline neural activity is adaptively adjusted during the trial.

10. The method of claim 1, wherein the feedback is displayed less than 5 seconds after neural signals are recorded.

11. The method of claim 1, wherein the feedback is displayed throughout the trial and updated at an interval during the trial.

12. The method of claim 1, wherein the subject's performance is scored based on changes in slow cortical potentials in the neural activity relative to the baseline reflecting bilateral or unilateral hemispheric activity, or bilateral differences between the hemispheres.

13. The method of claim 1, wherein the subject forms a mental image without a prompt.

14. The method of claim 12, wherein the mental image wherein the mental image is created based on a memory of the subject.

15. The method of claim 1, further comprising after each trial of the neurofeedback training, instructing the subject to improve the score in the future.

16. The method of claim 1, wherein the cognitive function is related to visual attention, concentration, short-term memory, emotion, or creativity.

17. The method of claim 1, wherein the user fixates on a point while imagining the mental image.

18. The method of claim 17, wherein the point is a shape on a monitor.

19. The method of claim 1, where in the feedback is provided as a visual stimulus, an auditory stimulus, and/or a tactile stimulus.

20. The method of claim 1, wherein the feedback reflects a score illustrative of a success rate related to the visual working memory.

21. The method of claim 20, wherein the score reflects a uniqueness of the mental image or a familiarity of the mental image.

22. A system configured to engage a subject in a neurofeedback training trial, the system comprising:
a memory storing instructions; and
a processor configured to access the memory and execute the instructions to:
receive neural signals that quantify visual working memory related to a mental image from a plurality of scalp electrodes;
score the subject's performance based on neural activity related to a cognitive function during an induction period within the neural signals; and
provide feedback to the subject based on the scoring to inform the subject of a success rate related to the visual working memory,
wherein a cognitive function of the subject improves after the trial.

23. The system of claim 22, further comprising the plurality of scalp electrodes configured to be positioned in predefined locations on the subject's scalp to record the neural signals.

24. The system of claim 23, wherein the scalp electrodes comprise active electrodes and/or passive electrodes.

25. The system of claim 23, wherein each of the scalp electrodes is associated with a channel that is at least one of a posterior parietal channel or an occipital channel.
